(19) United States
(12) Patent Application Publication
(10) Pub. No.: US 2006/0194222 A1
(43) Pub. Date: Aug. 31, 2006

(54) Triplex probe compositions and methods for polynucleotide detection

(75) Inventors: Joseph A. Sorge, Del Mar, CA (US); Scott Happe, Austin, TX (US); Andrew Firmin, Jackson, WY (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 11/254,334

(22) Filed: Oct. 20, 2005

Related U.S. Application Data
(60) Provisional application No. 60/620,561, filed on Oct. 20, 2004.

(51) Int. Cl. C12Q 1/68; C07H 21/04

(52) U.S. Cl. 435/6; 536/25.32; 536/24.3

(57) ABSTRACT

The present invention provides composition and methods for the detection and measurement of target nucleic acids. The probes of the present invention, or triplex probes, comprise a complex of three oligonucleotide probes including: (1) a first oligonucleotide probe, (2) a second oligonucleotide probe, and (3) a bridging oligonucleotide probe. In most aspects of the invention, the first and second oligonucleotide probes preferentially hybridize to the bridging oligonucleotide in the absence of a target nucleic acid. The first oligonucleotide probe contains one member of an interactive pair of labels and the second oligonucleotide probe contains the other member of the interactive pair of labels. Separation of the first and second oligonucleotide probes (e.g., binding to target, cleavage of first, second, or bridging oligonucleotide) generates a detectable signal indicating the presence of a target nucleic acid.

### RELATED APPLICATION

This application claims the benefit of U.S. Provisional Application No. 60/620,561 filed on Oct. 20, 2004. The entire teachings of the above application is incorporated herein by reference.

### FIELD OF INVENTION

The invention relates in general to compositions for detecting or measuring a target nucleic acid sequence.

### BACKGROUND OF THE INVENTION

Techniques for polynucleotide detection have found widespread use in basic research, diagnostics, and forensics. Polynucleotide detection can be accomplished by a number of methods. Most methods rely on the use of the polymerase chain reaction (PCR) to amplify the amount of target DNA.

The TaqMan™ assay is a homogenous assay for detecting polynucleotides (U.S. Pat. No. 5,723,591). In this assay, two PCR primers flank a central probe oligonucleotide. The probe oligonucleotide contains two fluorescent moieties. During the polymerization step of the PCR process, the polymerase cleaves the probe oligonucleotide. The cleavage causes the two fluorescent moieties to become physically separated, which causes a change in the wavelength of the fluorescent emission. As more PCR product is created, the intensity of the novel wavelength increases.

Molecular beacons are an alternative to TaqMan™ (U.S. Pat. Nos. 6,277,607; 6,150,097; 6,037,130) for the detection of polynucleotides. Molecular beacons are oligonucleotide hairpins which undergo a conformational change upon binding to a perfectly matched template. The conformational change of the oligonucleotide increases the physical distance between a fluorophore moiety and a quencher moiety present on the oligonucleotide. This increase in physical distance causes the effect of the quencher to be diminished, thus increasing the signal derived from the fluorophore.

U.S. Pat. No. 6,174,670B1 discloses methods of monitoring hybridization during a polymerase chain reaction which are achieved with rapid thermal cycling and use of double stranded DNA dyes or specific hybridization probes in the presence of a fluorescence resonance energy transfer pair—fluorescein and Cy5.3 or Cy5.5. The method amplifies the target sequence by polymerase chain reaction in the presence of two nucleic acid probes that hybridize to adjacent regions of the target sequence, one of the probes being labeled with an acceptor fluorophore and the other probe labeled with a donor fluorophore of a fluorescence energy transfer pair such that upon hybridization of the two probes with the target sequence, the donor fluorophore interacts with the acceptor fluorophore to generate a detectable signal. The sample is then excited with light at a wavelength absorbed by the donor fluorophore and the fluorescent emission from the fluorescence energy transfer pair is detected for the determination of that target amount.

### SUMMARY OF THE INVENTION

The present invention provides composition and methods for the detection and measurement of target nucleic acids. The probes of the present invention, or triplex probes, comprise a complex of three oligonucleotide probes including: (1) a first oligonucleotide probe, (2) a second oligonucleotide probe, and (3) a bridging oligonucleotide probe. In most aspects of the invention, the first and second oligonucleotide probes preferentially hybridize to the bridging oligonucleotide in the absence of a target nucleic acid. The first oligonucleotide probe contains one member of an interactive pair of labels and the second oligonucleotide probe contains the other member of the interactive pair of labels. Separation of the first and second oligonucleotide probes (e.g., binding to target, cleavage of first, second, or bridging oligonucleotide) generates a detectable signal indicating the presence of a target nucleic acid.

In a first aspect of the invention, the invention provides for an oligonucleotide probe complex of a first oligonucleotide probe, a second oligonucleotide probe and a bridging oligonucleotide probe. At least one of the first or second oligonucleotide probes binds to a target nucleic acid. Both the first and second oligonucleotide probes have a member of an interactive pair of labels. The bridging oligonucleotide probe binds to at least a portion of each of the first and second oligonucleotide probes, and maintains the members of the interactive pair of labels in close proximity.

In one embodiment of the oligonucleotide probe complex, the interactive pair of labels comprise a fluorophore and a quencher. The fluorophore or quencher can be attached to a 3′ nucleotide of the first oligonucleotide probe and the other of the fluorophore or the quencher can be attached to a 5′ nucleotide of the second oligonucleotide probe. The interactive pair of labels may be separated by 0 to 15 nucleotides, preferably between 0 to 5 nucleotides. The fluorophore may be a FAM, R110, TAMRA, R6G, CAL Fluor Red 610, CAL Fluor Gold 540, or CAL Fluor Orange 560 and the quencher may be a DABCYL, BHQ-1, BHQ-2, and BHQ-3. In some embodiments, the detectable signal increases upon hybridization or cleavage of the first and second oligonucleotide probes by at least 2 fold.

The oligonucleotide probe complex can be used for detecting a target nucleic acid in a sample by contacting the sample with the oligonucleotide probe complex and determining the presence of the target nucleic acid in said sample. A change in the intensity of the signal is indicative of the presence of the target nucleic acid.

The invention also provides for a method of detecting a target nucleic acid in a sample by providing a PCR mixture which includes the oligonucleotide probe complex, a nucleic acid polymerase, a 5′ to 3′ nuclease and a pair of primers. The PCR mixture is contacted with the sample to produce a PCR sample mixture and the PCR sample mixture is incubated, to allow amplification of the target nucleic acid and cleavage of said first and/or second oligonucleotide probes with the 5′ to 3′ nuclease. The generation of a detectable signal is indicative of the presence of the target nucleic acid in said sample.

In one embodiment of the method, the nucleic acid polymerase substantially lacks 5′ to 3′ exonuclease activity. The nucleic acid polymerase can be a DNA polymerase and the 5′ to 3′ nuclease may be a FEN nuclease.

In another aspect of the invention, the oligonucleotide probe complex includes a first oligonucleotide probe, a second oligonucleotide probe and a bridging oligonucleotide probe. The first oligonucleotide probe and the second oligonucleotide probe are attached to a member of an interactive pair of labels. At least one of the first or second oligonucleotide probes binds to a target nucleic acid through a primer region. The bridging oligonucleotide probe binds to at least a portion of each of the first and second oligonucleotide probes, and maintains the members of the interactive pair of labels in close proximity.

In one embodiment of the invention, the interactive pair of labels can be a fluorophore and a quencher and one of the fluorophore or the quencher is attached to a 3′ nucleotide of the second oligonucleotide probe and the other is attached to a 5′ nucleotide of the first oligonucleotide probe. The interactive pair of labels may be separated by 0 to 5 nucleotides. The fluorophore may be a FAM, R110, TAMRA, R6G, CAL Fluor Red 610, or CAL Fluor Gold 540, and CAL Fluor Orange 560 and the quencher may be a DABCYL, BHQ-1, BHQ-2, or BHQ-3. The detectable signal increases upon hybridization of the first oligonucleotide probe by at least 2 fold.

The invention also provides for a method of detecting a target nucleic acid in a sample by providing a PCR mixture which includes the oligonucleotide probe complex, a nucleic acid polymerase, and a primer. The PCR mixture is contacted with the sample to produce a PCR sample mixture and the PCR sample mixture is incubated, to allow amplification of the target nucleic acid. The generation of a detectable signal is indicative of the presence of the target nucleic acid in said sample.

In another aspect, the invention provides an oligonucleotide probe complex, comprising a first oligonucleotide probe, a second oligonucleotide probe and a bridging oligonucleotide probe. The first and second oligonucleotide probes are attached to a member of an interactive pair of labels. The bridging oligonucleotide probe binds to, at least a portion of, each of the first and second oligonucleotide probes. The bridging oligonucleotide probe binds to a target nucleic acid through a primer region. The bridging oligonucleotide probe also, maintains members of the interactive pair of labels in close proximity.

In one embodiment of this aspect of the invention, the interactive pair of labels is a fluorophore and a quencher. The fluorophore or the quencher may be attached to a 3′ nucleotide of the second oligonucleotide probe and the other of the fluorophore or the quencher may be attached to a 5′ nucleotide of the first oligonucleotide probe. The interactive pair of labels can be a fluorophore and a quencher and one of the fluorophore or the quencher is attached to a 3′ nucleotide of the second oligonucleotide probe and the other is attached to a 5′ nucleotide of the first oligonucleotide probe. The interactive pair of labels may be separated by 0 to 5 nucleotides. The fluorophore may be a FAM, R110, TAMRA, R6G, CAL Fluor Red 610, or CAL Fluor Gold 540, and CAL Fluor Orange 560 and the quencher may be a DABCYL, BHQ-1, BHQ-2, or BHQ-3. The detectable signal increases upon hybridization of the first oligonucleotide probe by at least 2 fold.

The invention also provides for a method of detecting a target nucleic acid in a sample by providing a PCR mixture which includes the oligonucleotide probe complex, a nucleic acid polymerase, a 5′ to 3′ nuclease and a primer. The PCR mixture is contacted with the sample to produce a PCR sample mixture and the PCR sample mixture is incubated, to allow amplification of the target nucleic acid and cleavage of the first and/or second oligonucleotide probes. The generation of a detectable signal is indicative of the presence of the target nucleic acid in said sample.

In one embodiment of this method, the nucleic acid polymerase substantially lacks 5′ to 3′ exonuclease activity. The nucleic acid polymerase may be a DNA polymerase, and the 5′ to 3′ nuclease may be a FEN nuclease.

In another aspect, the invention provides an oligonucleotide probe complex, comprising a first oligonucleotide probe, a second oligonucleotide probe and a bridging oligonucleotide probe. The first oligonucleotide probe and a second oligonucleotide probes are attached to a member of an interactive pair of labels. The bridging oligonucleotide probe binds to at least a portion of each of the first and second oligonucleotide probes and also binds to a target nucleic acid. The bridging oligonucleotide probe, maintains the members of the interactive pair of labels in close proximity.

In one embodiment of this aspect of the invention, the interactive pair of labels is a fluorophore and a quencher. The fluorophore or the quencher may be attached to a 3′ nucleotide of the second oligonucleotide probe and the other of the fluorophore or the quencher may be attached to a 5′ nucleotide of the first oligonucleotide probe. The interactive pair of labels can be a fluorophore and a quencher and one of the fluorophore or the quencher is attached to a 3′ nucleotide of the second oligonucleotide probe and the other is attached to a 5′ nucleotide of the first oligonucleotide probe. The interactive pair of labels may be separated by 0 to 5 nucleotides. The fluorophore may be a FAM, R110, TAMRA, R6G, CAL Fluor Red 610, or CAL Fluor Gold 540, and CAL Fluor Orange 560 and the quencher may be a DABCYL, BHQ-1, BHQ-2, or BHQ-3. The detectable signal increases upon hybridization of the first oligonucleotide probe by at least 2 fold.

The invention also provides for a method of detecting a target nucleic acid in a sample by providing a PCR mixture which includes the oligonucleotide probe complex of the present aspect, a nucleic acid polymerase, a 5′ to 3′ nuclease and a primer. The PCR mixture is contacted with the sample to produce a PCR sample mixture and the PCR sample mixture is incubated, to allow amplification of the target nucleic acid and cleavage of the first and/or second oligonucleotide probes. The generation of a detectable signal is indicative of the presence of the target nucleic acid in said sample.

In one embodiment of this method, the nucleic acid polymerase substantially lacks 5′ to 3′ exonuclease activity. The nucleic acid polymerase may be a DNA polymerase, and the 5′ to 3′ nuclease may be a FEN nuclease.

Another aspect of the invention includes compositions. The compositions comprise a probe of the invention and a primer. In another embodiment, the compositions also includes a nucleic acid polymerase. The nucleic acid polymerase may be a DNA polymerase. The nucleic acid polymerase may substantially lack 5′ to 3′ exonuclease activity. In further embodiments of the composition, the composition further comprises a FEN nuclease.

In additional aspects of the invention, the probes are part of a kit for generating a signal indicative of the presence of a target nucleic acid sequence in a sample. The kits may include a nucleic acid polymerase substantially lacking 5′ to 3′ exonuclease activity, a suitable buffer, a FEN nuclease, a primer and packaging material therefor.

### BRIEF DESCRIPTION OF THE DRAWINGS

FIG. 1 shows three variations of oligonucleotide probe complexes of the invention used in Quantitative Polymerase Chain Reaction (QPCR). Each oligonucleotide probe complex comprises a bridging oligonucleotide probe (top strand); a first oligonucleotide probe which is complementary to the bridging oligonucleotide probe and a target nucleic acid, and has a fluorophore attached to the 5′ nucleotide (bottom right oligonucleotide strand); and a second oligonucleotide probe complementary to the bridging oligonucleotide with a quencher attached to the 3′ nucleotide (bottom left oligonucleotide strand).

FIG. 2 depicts the method of the oligonucleotide probe complex of FIG. 1 in a QPCR reaction.

FIG. 3 depicts another aspect of the oligonucleotide probe complex of the invention in which one of the oligonucleotide probes of the complex hybridizes to a target and acts as a primer.

FIG. 4 depicts another aspect of the oligonucleotide probe complex of the invention, in which the bridging oligonucleotide probe of the complex hybridizes to a target and acts as a primer.

FIG. 5 depicts another aspect of the oligonucleotide probe complex of the invention, in which the bridging oligonucleotide probe of the complex hybridizes to a target sequence and is incorporated into an amplicon.

FIG. 6 depicts detection of Streptococcus agalactiae genomic DNA within a sample, using a triplex probe and a pair of primers. FIG. 6A shows results of duplicate amplification reactions (filled circles), compared with a no-probe control (empty rectangles). FIG. 6B shows the effects of different probe concentrations of between 0 to 400 nM.

FIG. 7 depicts detection of CFTR using probe concentrations of 100 nM, 200 nM and 300 nM.

### DETAILED DESCRIPTION

Definitions

As used herein, a “polynucleotide” refers to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The term “polynucleotide” includes, without limitation, single- and double-stranded polynucleotide. The term “polynucleotide” as it is employed herein embraces chemically, enzymatically or metabolically modified forms of polynucleotide. “Polynucleotide” also embraces a short polynucleotide, often referred to as an oligonucleotide (e.g., a primer or a probe). A polynucleotide has a “5′-terminus” and a “3′-terminus” because polynucleotide phosphodiester linkages occur to the 5′ carbon and 3′ carbon of the pentose ring of the substituent mononucleotides. The end of a polynucleotide at which a new linkage would be to a 5′ carbon is its 5′ terminal nucleotide. The end of a polynucleotide at which a new linkage would be to a 3′ carbon is its 3′ terminal nucleotide. A terminal nucleotide, as used herein, is the nucleotide at the end position of the 3′- or 5′-terminus. As used herein, a polynucleotide sequence, even if internal to a larger polynucleotide (e.g., a sequence region within a polynucleotide), also can be said to have 5′- and 3′- ends.

As used herein, the term “oligonucleotide” refers to a short polynucleotide, typically less than or equal to 150 nucleotides long (e.g., between 5 and 150, preferably between 10 to 100, more preferably between 15 to 50 nucleotides in length). However, as used herein, the term is also intended to encompass longer or shorter polynucleotide chains. An “oligonucleotide” may hybridize to other polynucleotides, therefore serving as a probe for polynucleotide detection, or a primer for polynucleotide chain extension.

As used herein, the term “oligonucleotide probe complex” or “triplex probe” refers to a complex of three oligonucleotide probes including: (1) a first “oligonucleotide probe”, (2) a “second oligonucleotide probe”, and (3) a “bridging oligonucleotide probe”.

As used herein, the term “oligonucleotide probe” refer to the two oligonucleotide probes, of the present invention, that are complementary and hybridize to at least a portion of a “bridging oligonucleotide”. The first “oligonucleotide probe” contains one member of an “interactive pair of labels” while the second “oligonucleotide probe” contains the other member of an “interactive pair of labels”. The “oligonucleotide probe” of the present invention is ideally less than or equal to 100 nucleotides in length, typically less than or equal to 70 nucleotides, for example less than or equal to 60, 50, 40, 30, 20 or 10 nucleotides in length.

As used herein, the term “bridging oligonucleotide probe” refers to one of the three oligonucleotides that comprise the “oligonucleotide probe complex” or “triplex oligonucleotide” of the present invention. The “bridging oligonucleotide probe” is complementary to at least a portion of the two “oligonucleotide probes” of the present invention. The “bridging oligonucleotide probe” preferentially binds the two “oligonucleotide probes” in the absence of a target nucleic acid. The “bridging oligonucleotide probe” binds the two “oligonucleotide probes” such that the two “oligonucleotide probes” are in close proximity, thereby, in some embodiments, “quenching” the interactive pair of labels. The “bridging oligonucleotide probe” of the present invention is ideally less than or equal to 100 nucleotides in length, typically less than or equal to 70 nucleotides, for example less than or equal to 60, 50, 40 or 30 nucleotides in length.

As used herein, an “interactive pair of labels” and a “pair of interactive labels” refer to a pair of molecules which interact physically, optically or otherwise in such a manner as to permit detection of their proximity by means of a detectable signal. Examples of a “pair of interactive labels” include, but are not limited to, labels suitable for use in fluorescence resonance energy transfer (FRET)(Stryer, L. Ann. Rev. Biochem. 47, 819-846, 1978), scintillation proximity assays (SPA) (Hart and Greenwald, Molecular Immunology 16:265-267, 1979; U.S. Pat. No. 4,658,649), luminescence resonance energy transfer (LRET) (Mathis, G. Clin. Chem. 41, 1391-1397, 1995), direct quenching (Tyagi et al., Nature Biotechnology 16, 49-53, 1998), chemiluminescence energy transfer (CRET) (Campbell, A. K., and Patel, A. Biochem. J. 216, 185-194, 1983), bioluminescence resonance energy transfer (BRET) (Xu, Y., Piston D. W., Johnson, Proc. Natl. Acad. Sc., 96, 151-156, 1999), or excimer formation (Lakowicz, J. R. Principles of Fluorescence Spectroscopy, Kluwer Academic/Plenum Press, New York, 1999).

As used herein, the term “quencher” refers to a chromophoric molecule or part of a compound, which is capable of reducing the emission from a fluorescent donor when attached to or in proximity to the donor. Quenching may occur by any of several mechanisms including fluorescence resonance energy transfer, photoinduced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more.

As used herein, references to “fluorescence” or “fluorescent groups” or ”fluorophores” include luminescence and luminescent groups, respectively.

An “increase in fluorescence”, as used herein, refers to an increase in detectable fluorescence emitted by a fluorophore. An increase in fluorescence may result, for example, when the distance between a fluorophore and a quencher is increased, for example due to a cleavage reaction, such that the quenching is reduced. There is an “increase in fluorescence” when the fluorescence emitted by the fluorophore is increased by at least 2 fold, for example 2, 2.5, 3, 4, 5, 6, 7, 8, 10 fold or more.

As used herein, the term “hybridization” or “binding” is used in reference to the pairing of complementary (including partially complementary) polynucleotide strands. Hybridization and the strength of hybridization (i.e., the strength of the association between polynucleotide strands) is impacted by many factors well known in the art including the degree of complementarity between the polynucleotides, stringency of the conditions involved affected by such conditions as the concentration of salts, the melting temperature (Tm) of the formed hybrid, the presence of other components (e.g., the presence or absence of polyethylene glycol), the molarity of the hybridizing strands and the G:C content of the polynucleotide strands.

As used herein, when one polynucleotide or oligonucleotide is said to “hybridize” or “bind to” another polynucleotide, it means that there is some complementarity between the two polynucleotides or that the two polynucleotides form a hybrid under high stringency conditions. When one polynucleotide is said to not hybridize to another polynucleotide, it means that there is no sequence complementarity between the two polynucleotides or that no hybrid forms between the two polynucleotides at a high stringency condition.

As used herein, a “primer” refers to a type of oligonucleotide having or containing the length limits of an “oligonucleotide” as defined above, and having or containing a sequence complementary to a target polynucleotide, which hybridizes to the target polynucleotide through base pairing so to initiate an elongation (extension) reaction to incorporate a nucleotide into the oligonucleotide primer. The conditions for initiation and extension include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. The primer is preferably single-stranded for maximum efficiency in amplification. “Primers” useful in the present invention are generally between about 10 and 100 nucleotides in length, preferably between about 17 and 50 nucleotides in length, and most preferably between about 17 and 45 nucleotides in length. An “amplification primer” is a primer for amplification of a target sequence by primer extension. As no special sequences or structures are required to drive the amplification reaction, amplification primers for PCR may consist only of target binding sequences. A “primer region” is a region on a “oligonucleotide probe” or a “bridging oligonucleotide probe” which hybridizes to the target nucleic acid through base pairing so to initiate an elongation reaction to incorporate a nucleotide into the oligonucleotide primer.

As used herein, the term “complementary” refers to the concept of sequence complementarity between regions of two polynucleotide strands or between two regions of the same polynucleotide strand. It is known that an adenine base of a first polynucleotide region is capable of forming specific hydrogen bonds (“base pairing”) with a base of a second polynucleotide region which is antiparallel to the first region if the base is thymine or uracil. Similarly, it is known that a cytosine base of a first polynucleotide strand is capable of base pairing with a base of a second polynucleotide strand which is antiparallel to the first strand if the base is guanine. A first region of a polynucleotide is complementary to a second region of the same or a different polynucleotide if, when the two regions are arranged in an antiparallel fashion, at least one nucleotide of the first region is capable of base pairing with a base of the second region. Therefore, it is not required for two complementary polynucleotides or oligonucleotides to base pair at every nucleotide position. “Complementary” refers to a first polynucleotide that is 100% or “fully” complementary to a second polynucleotide and thus forms a base pair at every nucleotide position. “Complementary” also refers to a first polynucleotide that is not 100% complementary or is partially complementary (e.g., 90%, or 80% or 70% complementary) and contains mismatched nucleotides at one or more nucleotide positions. In one embodiment, two complementary polynucleotides are capable of hybridizing to each other under high stringency hybridization conditions. For example, for membrane hybridization (e.g., Northern hybridization), high stringency hybridization conditions are defined as incubation with a radiolabeled probe in 5× SSC, 5× Denhardt's solution, 1% SDS at 65° C. Stringent washes for membrane hybridization are performed as follows: the membrane is washed at room temperature in 2× SSC/0.1% SDS and at 65° C. in 0.2× SSC/0.1% SDS, 10 minutes per wash, and exposed to film.

As used herein, a polynucleotide “isolated” from a sample is a naturally occurring polynucleotide sequence within that sample which has been removed from its normal cellular (e.g., chromosomal) environment. Thus, an “isolated” polynucleotide may be in a cell-free solution or placed in a different cellular environment.

As used herein, the term “amount” refers to an amount of a target polynucleotide in a sample, e.g., measured in μg, μmol or copy number. The abundance of a polynucleotide in the present invention is measured by the fluorescence intensity emitted by such polynucleotide, and compared with the fluorescence intensity emitted by a reference polynucleotide, i.e., a polynucleotide with a known amount.

As used herein, the term “homology” refers to the optimal alignment of sequences (either nucleotides or amino acids), which may be conducted by computerized implementations of algorithms. “Homology”, with regard to polynucleotides, for example, may be determined by analysis with BLASTN version 2.0 using the default parameters. A “probe which shares no homology with another polynucleotide” refers to that the homology between the probe and the polynucleotide, as measured by BLASTN version 2.0 using the default parameters, is no more than 55%, e.g., less than 50%, or less than 45%, or less than 40%, or less than 35%, in a contiguous region of 20 nucleotides or more.

As used herein, “nucleic acid polymerase” refers to an enzyme that catalyzes the polymerization of nucleoside triphosphates. Generally, the enzyme will initiate synthesis at the 3′-end of the primer annealed to the target sequence, and will proceed in the 5′-direction along the template, and if possessing a 5′ to 3′ nuclease activity, hydrolyzing intervening, annealed probe to release both labeled and unlabeled probe fragments, until synthesis terminates. Known DNA polymerases include, for example, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase and Pyrococcus furiosus (Pfu) DNA polymerase.

As used herein, “5′ to 3′ exonuclease activity” or “5′→3′ exonuclease activity” refers to that activity of a template-specific nucleic acid polymerase e.g. a 5′-3′ exonuclease activity traditionally associated with some DNA polymerases whereby mononucleotides or oligonucleotides are removed from the 5′ end of a polynucleotide in a sequential manner, (i.e., E. coli DNA polymerase I has this activity whereas the Klenow (Klenow et al., 1970, Proc. Natl. Acad. Sci., USA, 65:168) fragment does not, (Klenow et al., 1971, Eur. J. Biochem., 22:371)), or polynucleotides are removed from the 5′ end by an endonucleolytic activity that may be inherently present in a 5′ to 3′ exonuclease activity.

As used herein, the phrase “substantially lacks 5′ to 3′ exonuclease activity” or “substantially lacks 5′→3′ exonuclease activity” means having less than 10%, 5%, 1%, 0.5%, or 0.1% of the activity of a wild type enzyme. The phrase “lacking 5′ to 3′ exonuclease activity” or “lacking 5′→3′ exonuclease activity” means having undetectable 5′ to 3′ exonuclease activity or having less than about 1%, 0.5%, or 0.1% of the 5′ to 3′ exonuclease activity of a wild type enzyme. 5′ to 3′ exonuclease activity may be measured by an exonuclease assay which includes the steps of cleaving a nicked substrate in the presence of an appropriate buffer, for example 10 mM Tris-HCl (pH 8.0), 10 mM MgCl₂ and 50 μg/ml bovine serum albumin) for 30 minutes at 60° C., terminating the cleavage reaction by the addition of 95% formamide containing 10 mM EDTA and 1 mg/ml bromophenol blue, and detecting nicked or un-nicked product.

In some embodiments of the invention, nucleic acid polymerases useful according to the invention substantially lack 5′ to 3′ exonuclease activity and include but are not limited to exo-Pfu DNA polymerase (a mutant form of Pfu DNA polymerase that substantially lacks 3′ to 5′ exonuclease activity, Cline et al., 1996, Nucleic Acids Research, 24: 3546; U.S. Pat. No. 5,556,772; commercially available from Stratagene, La Jolla, Calif. Catalogue #600163), exo-Tma DNA polymerase (a mutant form of Tma DNA polymerase that substantially lacks 3′ to 5′ exonuclease activity), exo-Tli DNA polymerase (a mutant form of Tli DNA polymerase that substantially lacks 3′ to 5′ exonuclease activity; commercially available from New England Biolabs, (Beverly, Mass.; Cat #257)), exo-E. coli DNA polymerase (a mutant form of E. coli DNA polymerase that substantially lacks 3′ to 5′ exonuclease activity) exo-Klenow fragment of E. coli DNA polymerase I (Stratagene, Cat #600069), exo-T7 DNA polymerase (a mutant form of T7 DNA polymerase that substantially lacks 3′ to 5′ exonuclease activity), exo-KOD DNA polymerase (a mutant form of KOD DNA polymerase that substantially lacks 3′ to 5′ exonuclease activity), exo-JDF-3 DNA polymerase (a mutant form of JDF-3 DNA polymerase that substantially lacks 3′ to 5′ exonuclease activity), exo-PGB-D DNA polymerase (a mutant form of PGB-D DNA polymerase that substantially lacks 3′ to 5′ exonuclease activity) New England Biolabs, Cat. #259, Tth DNA polymerase, Taq DNA polymerase (e.g., Cat. Nos. 600131, 600132, 600139, Stratagene); UlTma (N-truncated) Thermatoga martima DNA polymerase; Klenow fragment of DNA polymerase I, 9° Nm DNA polymerase (discontinued product from New England Biolabs, Beverly, Mass.), “3′-5′ exo reduced” mutant (Southworth et al., 1996, Proc. Natl. Acad. Sci 93:5281) and Sequenase™ (USB, Cleveland, Ohio). The polymerase activity of any of the above enzyme can be defined by means well known in the art. One unit of DNA polymerase activity, according to the subject invention, is defined as the amount of enzyme which catalyzes the incorporation of 10 nmoles of total dNTPs into polymeric form in 30 minutes at optimal temperature.

“Primer extension reaction” or “synthesizing a primer extension” means a reaction between a target-primer hybrid and a nucleotide which results in the addition of the nucleotide to a 3′-end of the primer such that the incorporated nucleotide is complementary to the corresponding nucleotide of the target polynucleotide. Primer extension reagents typically include (i) a polymerase enzyme; (ii) a buffer; and (iii) one or more extendible nucleotides.

As used herein, “polymerase chain reaction” or “PCR” refers to an in vitro method for amplifying a specific polynucleotide template sequence. The PCR reaction involves a repetitive series of temperature cycles and is typically performed in a volume of 50-100 μl. The reaction mix comprises dNTPs (each of the four deoxynucleotides dATP, dCTP, dGTP, and dTTP), primers, buffers, DNA polymerase, and polynucleotide template. One PCR reaction may consist of 5 to 100 “cycles” of denaturation and synthesis of a polynucleotide molecule. The PCR process is described in U.S. Pat. Nos. 4,683,195 and 4,683,202, the disclosures of which are incorporated herein by reference.

As used herein a “nuclease” or a “cleavage agent” refers to an enzyme that is specific for, that is, cleaves a “cleavage structure” according to the invention and is not specific for, that is, does not substantially cleave either a probe or a primer that is not hybridized to a target nucleic acid, or a target nucleic acid that is not hybridized to a probe or a primer. The term “nuclease” includes an enzyme that possesses 5′ endonucleolytic activity for example a DNA polymerase, e.g. DNA polymerase I from E. coli, and DNA polymerase from Thermus aquaticus (Taq), Thermus thermophilus (Tth), Pyrococcus furiosus (Pfu) and Thermus flavus (Tfl). The term “nuclease” also embodies FEN nucleases. Nucleases are described in U.S. Pat. Nos. 6,528,254, 6,548,250 and 6,090,543 all of which are herein incorporated by reference in their entireties.

As used herein, a “cleavage structure” refers to a polynucleotide structure comprising at least a duplex nucleic acid having a single stranded region comprising a flap, a loop, a single-stranded bubble, a D-loop, a nick or a gap. A cleavage structure can be created by a nucleic acid polymerase. Cleavage structures are described in U.S. Pat. Nos. 6,528,254 and 6,548,250 both of which are herein incorporated by reference in their entireties.

The term “FEN nuclease” encompasses any enzyme that possesses 5′ exonuclease and/or an endonuclease activity. The term “FEN nuclease” also embodies a 5′ flap-specific nuclease. A nuclease or cleavage agent according to the invention includes but is not limited to a FEN nuclease enzyme derived from Archaeglobus fulgidus, Methanococcus jannaschii, Pyrococcus furiosus, human, and mouse or Xenopus laevis. A nuclease according to the invention also includes Saccharomyces cerevisiae RAD27, and Schizosaccharomyces pombe RAD2, Pol I DNA polymerase associated 5′ to 3′ exonuclease domain, (e.g. E. coli, Thermus aquaticus (Taq), Thermus flavus (Tfl), Bacillus caldotenax (Bca), Streptococcus pneumoniae) and phage functional homologs of FEN including but not limited to T5 5′ to 3′ exonuclease, T7 gene 6 exonuclease and T3 gene 6 exonuclease. Preferably, only the 5′ to 3′ exonuclease domains of Taq, Tfl and Bca FEN nuclease are used. The term “nuclease” does not include RNAse H. A FEN enzyme and its method of use are described in U.S. Pat. Nos. 6,528,254 and 6,548,250, the disclosures of which are incorporated herein by reference.

As used herein, “Tₘ” and “melting temperature” are interchangeable terms which are the temperature at which 50% of a population of double-stranded polynucleotide molecules becomes dissociated into single strands. The equation for calculating the Tm of polynucleotides is well known in the art. For example, the Tₘ may be calculated by the following equation: Tₘ=69.3+0.41×(G+C)%−650/L, wherein L is the length of the probe in nucleotides. The Tₘ of a hybrid polynucleotide may also be estimated using a formula adopted from hybridization assays in 1 M salt, and commonly used for calculating Tₘ for PCR primers: [(number of A+T)×2° C.+(number of G+C)×4° C.], see, for example, C. R. Newton et al. PCR, 2^{nd} Ed., Springer-Verlag (New York: 1997), p. 24. Other more sophisticated computations exist in the art, which take structural as well as sequence characteristics into account for the calculation of Tₘ. A calculated Tₘ is merely an estimate; the optimum temperature is commonly determined empirically.

A “nucleotide analog”, as used herein, refers to a nucleotide in which the pentose sugar and/or one or more of the phosphate esters is replaced with its respective analog. Exemplary pentose sugar analogs are those previously described in conjunction with nucleoside analogs. Exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., including any associated counterions, if present. Also included within the definition of “nucleotide analog” are nucleobase monomers which can be polymerized into polynucleotide analogs in which the DNA/RNA phosphate ester and/or sugar phosphate ester backbone is replaced with a different type of linkage.

As used herein, the term “in close proximity,” refers to the relative distance to which two target-hybridizing probes hybridize to the same strand of a “bridging oligonucleotide probe”, the distance being sufficient to permit the interaction of labels on the two “oligonucleotide probes”. The distance between the two hybridization sites is less than 50 nucleotides, preferably less than 30 nucleotides, more preferably less than 5 nucleotides, for example, less than 1 nucleotide.

As used herein, the term “sample” refers to a biological material which is isolated from its natural environment and containing a polynucleotide. A “sample” according to the invention may consist of purified or isolated polynucleotide, or it may comprise a biological sample such as a tissue sample, a biological fluid sample, or a cell sample comprising a polynucleotide. A biological fluid includes blood, plasma, sputum, urine, cerebrospinal fluid, lavages, and leukophoresis samples. A sample of the present invention may be a plant, animal, bacterial or viral material containing a target polynucleotide. Useful samples of the present invention may be obtained from different sources, including, for example, but not limited to, from different individuals, different developmental stages of the same or different individuals, different disease individuals, normal individuals, different disease stages of the same or different individuals, individuals subjected to different disease treatment, individuals subjected to different environmental factors, individuals with predisposition to a pathology, individuals with exposure to an infectious disease (e.g., HIV). Useful samples may also be obtained from in vitro cultured tissues, cells, or other polynucleotide containing sources. The cultured samples may be taken from sources including, but are not limited to, cultures (e.g., tissue or cells) cultured in different media and conditions (e.g., pH, pressure, or temperature), cultures (e.g., tissue or cells) cultured for different period of length, cultures (e.g., tissue or cells) treated with different factors or reagents (e.g., a drug candidate, or a modulator), or cultures of different types of tissue or cells.

Description

The present invention provides oligonucleotide probe complexes for polynucleotide detection. The triplex probes of the present invention comprise three oligonucleotide probes which include: (1) a first oligonucleotide probe, (2) a second oligonucleotide probe, and (3) a bridging oligonucleotide probe. The first and second oligonucleotide probes are complementary to and hybridize with the bridging oligonucleotide in the absence of a target nucleic acid. In most embodiments, the first or second oligonucleotide probe does not hybridize in the presence of the target nucleic acid. As discussed in more detail below, in some embodiments, the first and second oligonucleotide probes either comprise additional nucleic acid sequences which hybridize to the target nucleic acid but not the bridging oligonucleotide, or the target nucleic acid has a higher degree of complementarity to the oligonucleotide probes than the oligonucleotide probes have to the bridging oligonucleotide. In other embodiments, the bridging oligonucleotide probe is complementary, and thus hybridizes, to the target nucleic acid. In some embodiments, the first and/or second oligonucleotide probe is complementary to a target nucleic acid and hybridizes to the target. The first oligonucleotide probe contains one member of an interactive pair of labels and the second oligonucleotide probe contains the other member of the interactive pair of labels. In some embodiments, the interactive pair of labels is a quencher and a fluorophore. When the first and second oligonucleotide probes are bound to the bridging oligonucleotide the interactive pair of labels are within close proximity of one another such that the interactive pair of labels interact. A detectable signal is generated by one or both members of the interactive pair of labels when the interactive pair of labels are not within close proximity, e.g., hybridization of target, cleavage by a 5′ to 3′ exonuclease. In a preferred embodiment, the first member of an interactive pair of labels is attached to the 3′ end nucleotide of one oligonucleotide probe and the second member of an interactive pair of labels is attached to the 5′ end of the second oligonucleotide probe. In other embodiments, the members of the interactive pair of labels are incorporated or attached to the non-terminal or internal nucleotides of the oligonucleotide probes.

According to the present invention, the oligonucleotide probe can comprise natural, non-natural nucleotides and analogs. The probe may be a nucleic acid analog or chimera comprising nucleic acid and nucleic acid analog monomer units, such as 2-aminoethylglycine. For example, part or all of the probe may be PNA or a PNA/DNA chimera.

The probe of the present invention is ideally less than 150 nucleotides in length, typically less than 100 nucleotides, for example less than 80, 70, 60 or 50 nucleotides in length. Preferably, the probe of the invention is between 10 and 60 nucleotides in length, more preferably between 15 and 45, and most preferably between 20 and 40 nucleotides in length.

Preferably the triplex probe system is used to monitor or detect the presence of a target DNA in a nucleic acid amplification reaction. The method, according to the invention, is performed using typical reaction conditions for standard polymerase chain reaction (PCR), with the exception that two temperature cycles are performed: one, a high temperature denaturation step (generally between 90° C. and 96° C.), typically between 1 and 30 seconds, and a combined annealing/extension step (anywhere between 50° C. and 65° C., depending on the annealing temperature of the probe and primer), usually between 10 and 90 seconds. The reaction mixture, also referred to as the “PCR mixture”, contains a nucleic acid, a nucleic acid polymerase as described above, the oligonucleotide probe complex of the present invention, suitable buffer and salts, and in some embodiments a FEN nuclease. The reaction can be performed in any thermocycler commonly used for PCR. However, preferred are cyclers with real-time fluorescence measurement capabilities, including instruments capable of measuring real-time including Taq Man 7700 AB (Applied Biosystems, Foster City, Calif.), Rotorgene 2000 (Corbett Research, Sydney, Australia), LightCycler (Roche Diagnostics Corp, Indianapolis, Ind.), iCycler (Biorad Laboratories, Hercules, Calif.) and Mx4000 (Stratagene, La Jolla, Calif.).

Use of a labeled probe generally in conjunction with the amplification of a target polynucleotide, for example, by PCR, e.g., is described in many references, such as Innis et al., editors, PCR Protocols (Academic Press, New York, 1989); Sambrook et al., Molecular Cloning, Second Edition (Cold Spring Harbor Laboratory, New York, 1989), all of which are hereby incorporated herein by reference. In some embodiments, the binding site of the probe is located between the PCR primers used to amplify the target polynucleotide. In other embodiments, the oligonucleotide probe complex of the invention acts as a primer. In another embodiment, the oligonucleotide probe complex of the invention binds to a target nucleic acid present in a primer incorporated into the amplicon. Preferably, PCR is carried out using Taq DNA polymerase, e.g., Amplitaq (Perkin-Elmer, Norwalk, Conn.), or an equivalent thermostable DNA polymerase, and the annealing temperature of the PCR is about 5° C. -10° C. below the melting temperature of the oligonucleotide probes employed.

The following descriptions further, identify various aspects of the probes of the invention.

Triplex Probes in Which One or Both Oligonucleotide Probes Hybridize to a Target Nucleic Acid

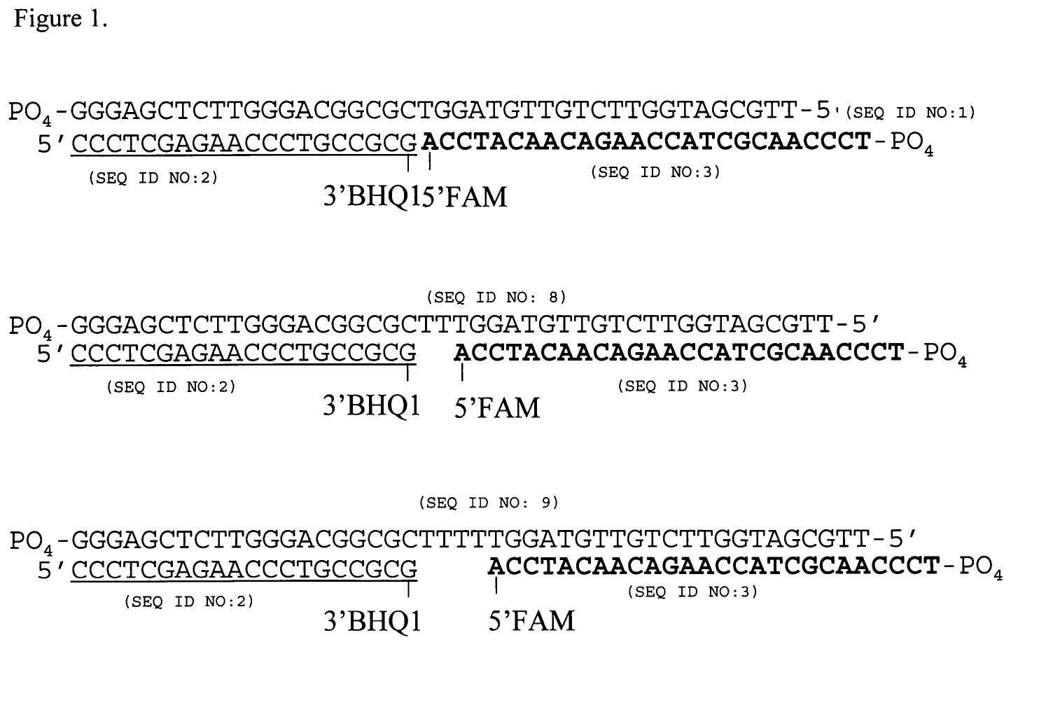

FIG. 1 describes one aspect of the present invention. As indicated in FIG. 1, the triplex oligonucleotide probe comprises three oligonucleotide sequences. The top oligonucleotide sequence or bridging oligonucleotide probe and the bottom two oligonucleotide sequences or the first and second oligonucleotide probes. The bridging oligonucleotide is complementary to the first and second oligonucleotide probe sequences. In some embodiments, the bridging oligonucleotide probe is fully complementary to the two oligonucleotide probe sequences. In alternative embodiments, the bridging oligonucleotide probe is partially complementary to the two oligonucleotide probe sequences. In some embodiments, the bridging oligonucleotide contains a spacer region between the oligonucleotide probe sequences. In other embodiments, the spacer region separates the interactive pair of labels, bound to the first and second oligonucleotide sequences by 0 to 5 nucleotides. In a preferred embodiment, there is no spacer region between the oligonucleotide probe sequences.

One or both oligonucleotide probe sequences are complementary to a target nucleic acid. In a preferred embodiment, a single oligonucleotide probe sequence is complementary to the target nucleic acid. In a further embodiment, the reporter oligonucleotide probe, or oligonucleotide probe having a fluorophore, is complementary to the target nucleic acid.

The oligonucleotide probes which hybridize to the target nucleic acid have a target binding region. This region is complementary to the target nucleic acid sequence. The region of the target nucleic acid, which is complementary to the target binding sequence, is ideally located within 200 nucleotides downstream of (i.e., to the 3′ of) the primer binding site, typically within 150, 125, or 100 nucleotides.

The first oligonucleotide probe contains one member of an interactive pair of labels and the second oligonucleotide probe contains the other member of the interactive pair of labels. In some embodiments, the interactive pair of labels is a quencher and a fluorescer. When the first and second oligonucleotide probes are hybridized to the bridging oligonucleotide, the interactive pair of labels are within close proximity of one another such that the interactive pair of labels interact. A detectable signal is generated by one or both members of the interactive pair of labels when the interactive pair of labels are not within close proximity, e.g., hybridization of target, cleavage by a nuclease. In some embodiments, the first member of an interactive pair of labels is attached to the 3′ end nucleotide of one oligonucleotide probe and the second member of an interactive pair of labels is attached to the 5′ end of the second oligonucleotide probe. In other embodiments, the members of the interactive pair of labels are incorporated or attached to the non-terminal or internal nucleotides of the oligonucleotide probes. In a preferred embodiment, the reporter oligonucleotide probe is complementary to the target and hybridizes to said target. In one embodiment, the reporter oligonucleotide has a fluorophore attached to the 5′ end nucleotide.

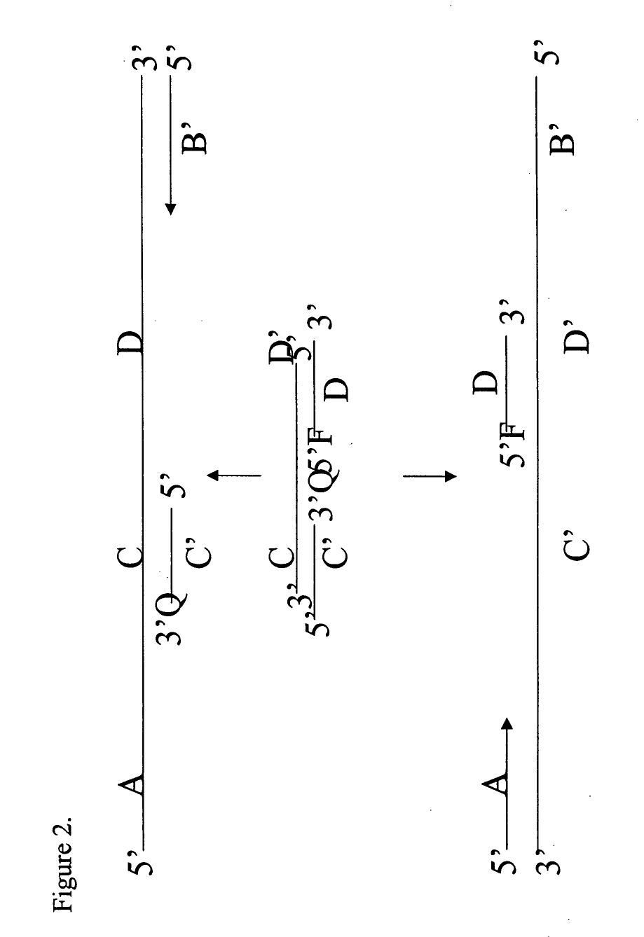

FIG. 2 illustrates a method of the oligonucleotide probe complex of the invention. In this embodiment, the two oligonucleotide probes disassociate from the bridging oligonucleotide probe and hybridize to their respective target nucleic acids, during a PCR reaction. In one embodiment, both oligonucleotide probes hybridize to their respective target nucleic acids. In another embodiment, a single oligonucleotide probe hybridizes to a target nucleic acid.

A detectable signal is generated by one member of the interactive pair of labels, when the two oligonucleotide probes are separated from each other, e.g., binding to their respective targets. In a preferred embodiment, a detectable signal is generated by cleavage of one or both the oligonucleotide probes when hybridized to the target. In a further preferred embodiment, the cleavage is by a FEN nuclease. Generation of a detectable signal by the cleavage of a cleavage structure is described in U.S. Pat. Nos. 6,528,254 and 6,548,250 both of which are herein incorporated by reference in their entireties.

Triplex Probe in Which One of the Oligonucleotide Probes Functions as a Primer

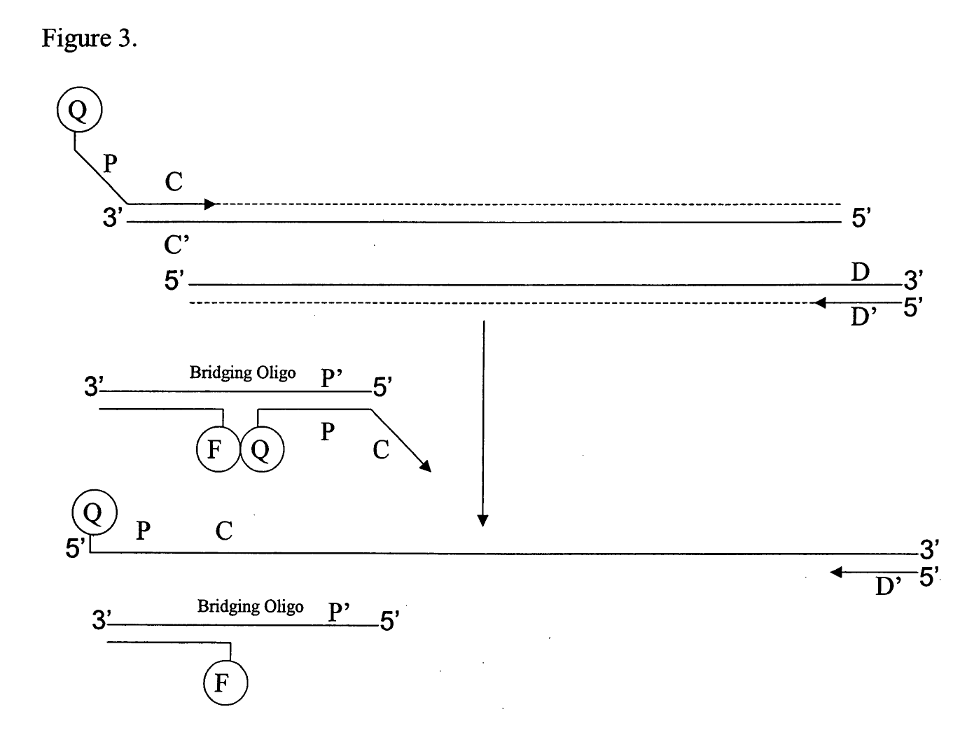

FIG. 3 describes another aspect of the present invention. As indicated in FIG. 3 the triplex probe comprises three oligonucleotide sequences. The top oligonucleotide sequence or bridging oligonucleotide probe and the bottom two oligonucleotide sequences or the first and second oligonucleotide probes. The bridging oligonucleotide is complementary to the first and second oligonucleotide probe sequences. In some embodiments, the bridging oligonucleotide probe is fully complementary to the two oligonucleotide probe sequences. In alternative embodiments, the bridging oligonucleotide probe is partially complementary to the two oligonucleotide probe sequences. In some embodiments, the bridging oligonucleotide contains a spacer region between the oligonucleotide probe sequences. In some embodiments, the spacer region separates the interactive pair of labels, bound to the first and second oligonucleotide sequences by 0 to 5 nucleotides. In a preferred embodiment, there is no spacer region between the oligonucleotide probe sequences.

The first oligonucleotide probe or primer probe, comprises a first segment which hybridizes to the bridging oligonucleotide, a second segment which hybridizes to the target sequence and functions as a primer, and a first member of an interactive pair of labels. In a preferred embodiment, the first segment is partially complementary to the bridging oligonucleotide sequence. The second oligonucleotide probe is complementary to and hybridizes with the bridging oligonucleotide, and contains a second member of an interactive pair of labels.

In a preferred embodiment, of this oligonucleotide probe complex, the detection reaction is conducted in a PCR assay format. The oligonucleotide probe complex of FIG. 3 is added to the PCR reaction mixture comprising a sense primer, target nucleic acid and dNTPs. The first oligonucleotide probe preferentially binds the target during the annealing step of the PCR protocol. The first oligonucleotide probe functions as a primer and the nucleic acid polymerase synthesizes a primer extension product, thus integrating the first oligonucleotide probe into the amplified product. Incorporation of the first oligonucleotide probe prevents the bridging oligonucleotide from hybridizing with the probe, thus separating the interactive pair of labels and generating a detectable signal.

Triplex Probe in Which the Bridging Oligonucleotide Probe Functions as a Primer

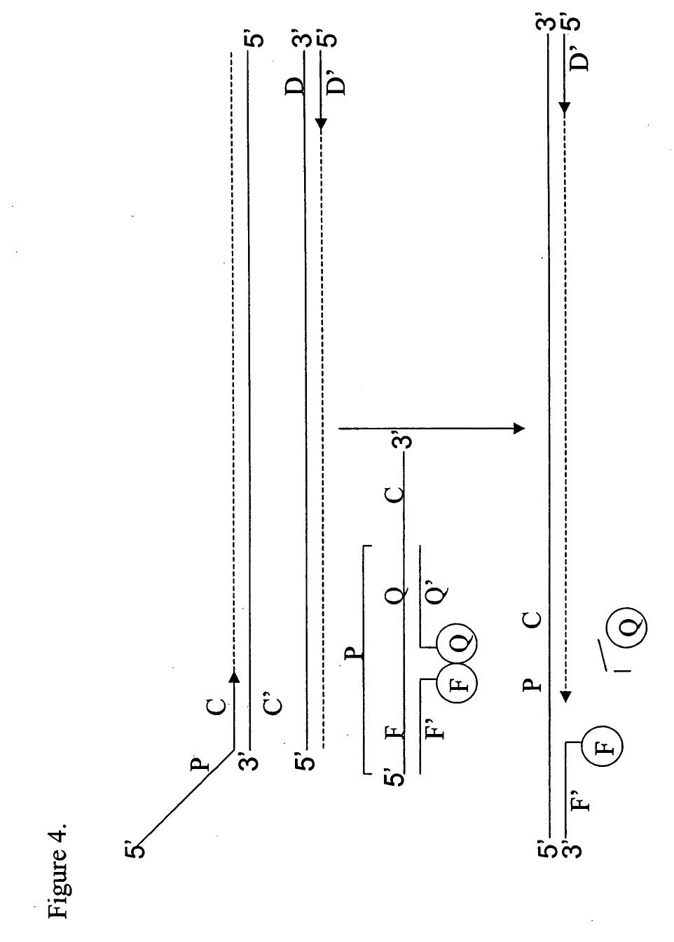

FIG. 4 describes another aspect of the present invention. As indicated in FIG. 4 the triplex probe comprises three oligonucleotide sequences. The top oligonucleotide sequence or bridging oligonucleotide probe and the bottom two oligonucleotide sequences or the first and second oligonucleotide probes. The bridging oligonucleotide has two segments. The first segment is complementary to the first and second oligonucleotide probes. The second segment is complementary to a target nucleic acid and acts as a primer when bound to the target. In some embodiments, the bridging oligonucleotide contains a spacer region between the oligonucleotide probe sequences. In some embodiments, the spacer region separates the interactive pair of labels, bound to the first and second oligonucleotide sequences, by 0 to 5 nucleotides. In a preferred embodiment, there is no spacer region between the oligonucleotide probe sequences.

In a preferred embodiment of the method of this oligonucleotide probe complex, the target detection reaction is conducted in a PCR assay format. The triplex probe of FIG. 4 is added to the PCR reaction mixture comprising a sense primer, target nucleic acid and dNTPs. The bridging oligonucleotide probe preferentially binds the target during the annealing step of the PCR protocol, and the first and second oligonucleotide probes hybridize to the bridging oligonucleotide. The bridging oligonucleotide probe acts as a primer and the nucleic acid polymerase synthesizes a primer extension product, incorporating the bridging oligonucleotide probe into the amplified product, while the first and second oligonucleotides remain bound. The sense strand is primed and extended by the nucleic acid polymerase. The extension of the sense strand creates a cleavage structure in one or both of the oligonucleotide probes, which is cleaved by a FEN nuclease. Cleavage of the first and/or second oligonucleotide probes, separates the members of the interactive pair of labels, generating a detectable signal. Generation of a detectable signal by the cleavage of a cleavage structure is described in U.S. Pat. Nos. 6,528,254 and 6,548,250 both of which are herein incorporated by reference in their entireties.

Triplex Probe in Which the Bridging Oligonucleotide Hybridizes to the Target.

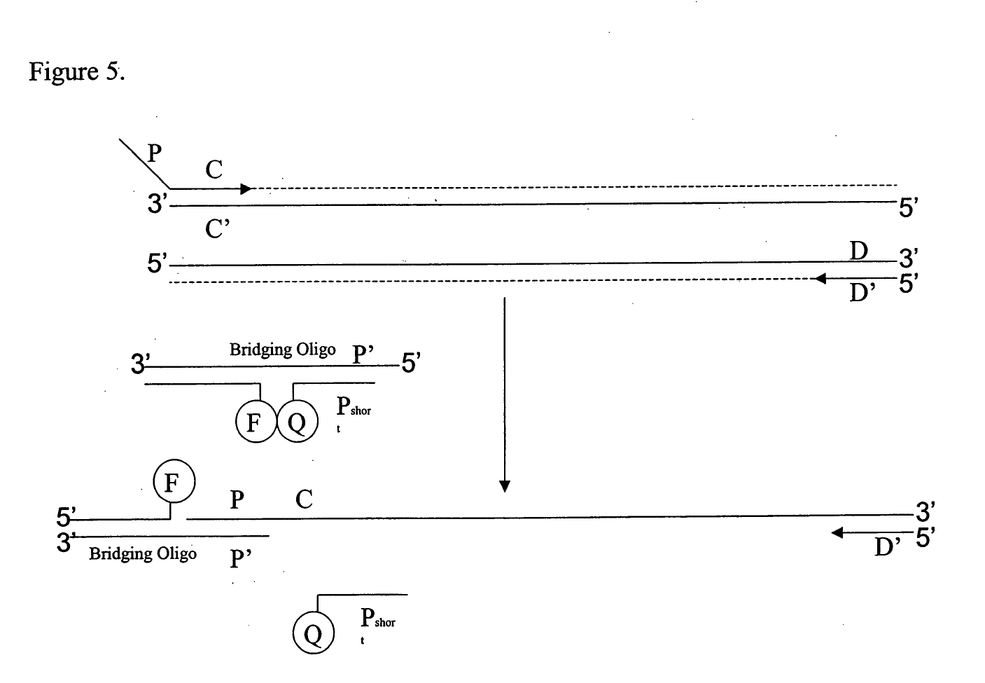

FIG. 5 describes another aspect of the present invention. As indicated in FIG. 5 the triplex probe comprises three oligonucleotide sequences. The top oligonucleotide sequence or bridging oligonucleotide probe and the bottom two oligonucleotide sequences or the first and second oligonucleotide probes. In some embodiments, the bridging oligonucleotide contains a spacer region between the oligonucleotide probe sequences. In some embodiments, the spacer region separates the interactive pair of labels, bound to the first and second oligonucleotide sequences, by 0 to 5 nucleotides. In a preferred embodiment, there is no spacer region between the oligonucleotide probe sequences.

The bridging oligonucleotide has a first bridging oligonucleotide sequence and a second bridging oligonucleotide sequence. The first bridging oligonucleotide sequence is complementary to the first oligonucleotide probe and the second oligonucleotide sequence is complementary to the second oligonucleotide probe and to a target oligonucleotide sequence. In a preferred embodiment, the second segment of the bridging oligonucleotide probe is longer than the second oligonucleotide probe.

In a preferred embodiment of the method of this probe of the invention, the target detection reaction is conducted in a PCR assay format. The triplex probe of FIG. 5 is added to the PCR reaction mixture comprising a set of primers, target nucleic acid and dNTPs. One primer contains a first segment which is complementary to and binds the target nucleic acid and a second segment which is complementary to and binds the bridging oligonucleotide probe. The primers hybridize to the target nucleic acid and are extended by a nucleic acid polymerase. This amplification reaction incorporates the bridging oligonucleotide binding region of the primer into the amplicon. The bridging oligonucleotide probe then, preferentially hybridizes with the target nucleic acid containing the incorporated bridging oligonucleotide probe sequence. Binding of the bridging oligonucleotide probe, prevents the second oligonucleotide probe from hybridizing to the bridging oligonucleotide, thus separating the interactive pair of labels, resulting in a detectable signal. In another embodiment, the PCR reaction mixture further comprises a FEN nuclease. The FEN nuclease will cleave the bound bridging oligonucleotide probe during polymerization of the sense strand. Cleavage destroys the second oligonucleotide probe's hybridization region in the bridging oligonucleotide, preventing rehybridization and enhancing the detectable signal.

Preparation of Primers and Probes

Probes and primers are typically prepared by biological or chemical synthesis, although they can also be prepared by biological purification or degradation, e.g., endonuclease digestion.

For short sequences such as probes and primers used in the present invention, chemical synthesis is frequently more economical as compared to biological synthesis. For longer sequences standard replication methods employed in molecular biology can be used such as the use of M13 for single stranded DNA as described by Messing, 1983, Methods Enzymol. 101:20-78. Chemical methods of polynucleotide or oligonucleotide synthesis include phosphotriester and phosphodiester methods (Narang, et al., Meth. Enzymol. (1979) 68:90) and synthesis on a support (Beaucage, et al., Tetrahedron Letters. (1981) 22:1859-1862) as well as phosphoramidate technique, Caruthers, M. H., et al., Methods in Enzymology (1988)154:287-314 (1988), and others described in “Synthesis and Applications of DNA and RNA,” S. A. Narang, editor, Academic Press, New York, 1987, and the references contained therein.

Oligonucleotide probes and primers can be synthesized by any method described above and other methods known in the art.

The bridging oligonucleotide binding sequence of the triplex probe, of the present invention, preferably has a higher Tm (e.g., at least 2° C., or 4° C., or 6° C., or 8° C., or 10° C., or 15° C., or 20° C., or higher) than the respective target binding sequence of the target-hybridizing probe.

Fluorophore

A pair of interactive labels useful for the invention can comprise a pair of FRET-compatible dyes, or a quencher-dye pair. In one embodiment, the pair comprises a fluorophore-quencher pair.

Oligonucleotide probes of the present invention permit monitoring of amplification reactions by fluorescence. They can be labeled with a fluorophore and quencher in such a manner that the fluorescence emitted by the fluorophore in intact probes is substantially quenched, whereas the fluorescence in cleaved or target hybridized oligonucleotide probes are not quenched, resulting in an increase in overall fluorescence upon probe cleavage or target hybridization. Furthermore, the generation of a fluorescent signal during real-time detection of the amplification products allows accurate quantitation of the initial number of target sequences in a sample.

A wide variety of fluorophores can be used, including but not limited to: 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6- carboxytetramethylrhodamine; Xanthylium, 9-(2,5-dicarboxyphenyl)-3, 6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl) azo)benzoic acid) Cy5 (Indodicarbocyanine-5) Cy3 (Indo-dicarbocyanine-3); and BODIPY FL (2,6-dibromo-4,4-difluoro-5,7 -dimethyl-4-bora-3a,4a-diaza-s-indacene-3-proprionic acid), Rox, as well as suitable derivatives thereof.

Quencher

The quencher can be any material that can quench at least one fluorescence emission from an excited fluorophore being used in the assay. There is a great deal of practical guidance available in the literature for selecting appropriate reporter-quencher pairs for particular probes, as exemplified by the following references: Clegg (1993, Proc. Natl. Acad. Sci., 90:2994-2998); Wu et al. (1994, Anal. Biochem., 218:1-13); Pesce et al., editors, Fluorescence Spectroscopy (1971, Marcel Dekker, New York); White et al., Fluorescence Analysis: A Practical Approach (1970, Marcel Dekker, New York); and the like. The literature also includes references providing exhaustive lists of fluorescent and chromogenic molecules and their relevant optical properties for choosing reporter-quencher pairs, e.g., Berlman, Handbook of Fluorescence Spectra of Aromatic Molecules, 2nd Edition (1971, Academic Press, New York); Griffiths, Colour and Constitution of Organic Molecules (1976, Academic Press, New York); Bishop, editor, Indicators (1972, Pergamon Press, Oxford); Haugland, Handbook of Fluorescent Probes and Research Chemicals (1992 Molecular Probes, Eugene) Pringsheim, Fluorescence and Phosphorescence (1949, Interscience Publishers, New York), all of which incorporated hereby by reference. Further, there is extensive guidance in the literature for derivatizing reporter and quencher molecules for covalent attachment via common reactive groups that can be added to an oligonucleotide, as exemplified by the following references, see, for example, Haugland (cited above); Ullman et al., U.S. Pat. No. 3,996,345; Khanna et al., U.S. Pat. No. 4,351,760, all of which hereby incorporated by reference.

A number of commercially available quenchers are known in the art, and include but are not limited to DABCYL, BHQ-1, BHQ-2, and BHQ-3. The BHQ quenchers are a new class of dark quenchers that prevent fluorescence until a hybridization event occurs. In addition, these new quenchers have no native fluorescence, virtually eliminating background problems seen with other quenchers. BHQ quenchers can be used to quench almost all reporter dyes and are commercially available, for example, from Biosearch Technologies, Inc (Novato, Calif.).

Attachment of Fluorophore and Quencher

In one embodiment of the invention, the fluorophore or quencher is attached to the 3′ nucleotide. In another embodiment of the invention, the fluorophore or quencher is attached to the 5′ nucleotide. In yet another embodiment, the fluorophore or quencher is internally attached to the oligonucleotide probe. In a preferred embodiment, one of said fluorophore or quencher is attached to the 5′ nucleotide of one oligonucleotide probe and the other of said fluorophore or quencher is attached to the 3′ nucleotide of the other oligonucleotide probe. Attachment can be made via direct coupling, or alternatively using a spacer molecule of between 1 and 5 atoms in length.

For the internal attachment of the fluorophore or quencher, linkage can be made using any of the means known in the art. Appropriate linking methodologies for attachment of many dyes to oligonucleotides are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565. All are hereby incorporated by reference.

The other of the fluorophore or quencher can be attached anywhere within the probe, preferably at a distance from the other of the fluorophore/quencher such that sufficient amount of quenching occurs when bound to the bridging oligonucleotide. Another preference is that the fluorophore and quencher be spaced sufficiently apart such that nuclease cleavage can occur readily between the two moieties during strand displacement. In one embodiment, the fluorophore and quencher are placed between 0 and 5 nucleotides when bound to said bridging oligonucleotide probe. In a preferred embodiment, the fluorophore and quencher are placed without any intervening nucleotides when bound to the bridging oligonucleotide probe.

When the oligonucleotide probe is intact, the moieties of the fluorophore/quencher pair are in a close, quenching relationship. For maximal quenching, the two moieties are ideally close to each other. In one embodiment, the quencher and fluorophore pair is positioned 30 or less nucleotides from each other. In a preferred embodiment, the pair is less than one nucleotide from each other.

### EXAMPLES

### Example 1

### Triplex Probe Design

Design of ideal probes for use according to the present invention uses the same rules as in designing PCR primers. The individual components of the probe are the quencher (Q) oligonucleotide, fluorophore (F) oligonucleotide and Bridging oligonucleotide, possibly with an attached primer. The Q and F oligonucleotides are designed with low free energy self dimer or cross hybridization possibilities (preferably less than or equal to 6 Kcal/mol for oligonucleotides approximately 25 bases or less). The bridging oligonucleotide is complementary to the F and Q probe sequences. The bridging oligonucleotide is checked to ensure no self dimer formation, as well as no cross dimer formation with other oligonucleotides in the mix. In some embodiments, the melting temperature for Q oligonucleotide, F oligonucleotide and primer regions are between 65 and 55° C., assuming an anneal/extension temperature of 60° C. Bridging oligonucleotides with MGBs, LNA and other modified nucleotides can be used. These oligonucleotides using synthetic nucleotides can be shortened while maintaining a high melting temperature.

### Example 2

### Use of Probe with Separate Primer Pair: Detection of Streptococcus agalactiae Sequences

The following experiment was performed to detect Streptococcus agalactiae genomic DNA within a sample.

Probe: a probe consisting of the following oligonucleotides were used for the assay.

BridgeS0 oligonucleotide was used as the bridging oligonucleotide:

5′ TTGCGATGGTTCTGTTGTAGGTCGCGGCAGGGTTCTCGAGGG 3′

Quencher oligonucleotide:

5′ CCCTCGAGAACCCTGCCGCG-BHQ1 3′

Fluorophore oligonucleotide:

5′ FAM-ACCTACAACAGAACCATCGCAACCCT 3′

The bridgeS0 oligonucleotide was designed to not contain any spacing nucleotides between the hybridization sites for the Quencher and Fluorophore oligonucleotides. The BHQ1 quencher was attached to the 3′ end of the quencher oligonucleotide and the FAM fluorophore was attached to the 5′ end of the fluorophore oligonucleotide such that, when the triplex was formed between these three oligonucleotides, the FAM fluorophore and BHQ1 quencher are in close proximity to each other, and therefore quenching the FAM signal:

```
PO₄ GGGAGCTCTTGGGACGGCGCTGGATGTTGTCTTGGTAGCGTT-5′
Bridge oligonucleotide
5′CCCTCGAGAACCCTGCCGCG ACCTACAACAGAACCATCGCAACCCT-
PO₄
      (FAM oligonucleotide) | | (Quencher
oligonucleotide)
                                        FAM BHQ
```

In this example, the individual oligonucleotides of the probe did not serve as templates for polymerase based primer extension. The fluorophore oligonucleotide can hybridize to the template between the forward and reverse GBS primer binding sites, at any position between 0 to 200 nucleotides from the primer binding site.

Amplification was performed in the presence of 100 nM each of the Quencher oligonucleotide (3′BHQ1) and the Fluorophore oligonucleotide (5°FAM), along with 500 nM of the BridgeS0 oligonucleotide. Results of other experiments suggest that that a molar excess of Bridge oligonucleotide is not necessary.

Amplification was carried out in 50 μl total reaction volume containing:

1× FullVelocity buffer (containing dUTP instead of dTTP),

400 nM Forward and Reverse GBS primes,

5 U Pfu V93R exo(-) polymerase,

200 ng FEN-1 endonuclease,

30 nM ROX reference dye, and

5000 copies Streptococcus agalactiae genomic DNA (template).

Components were mixed together and thermo-cycled on the M×3000p real-time PCR instrument: 1 cycle at 95° C. for 2 minutes, followed by 40 cycles of:

95° C. for 1 second

60° C. for 18 seconds

Fluorescence data was collected at the end of the 60° C. step of each cycle.

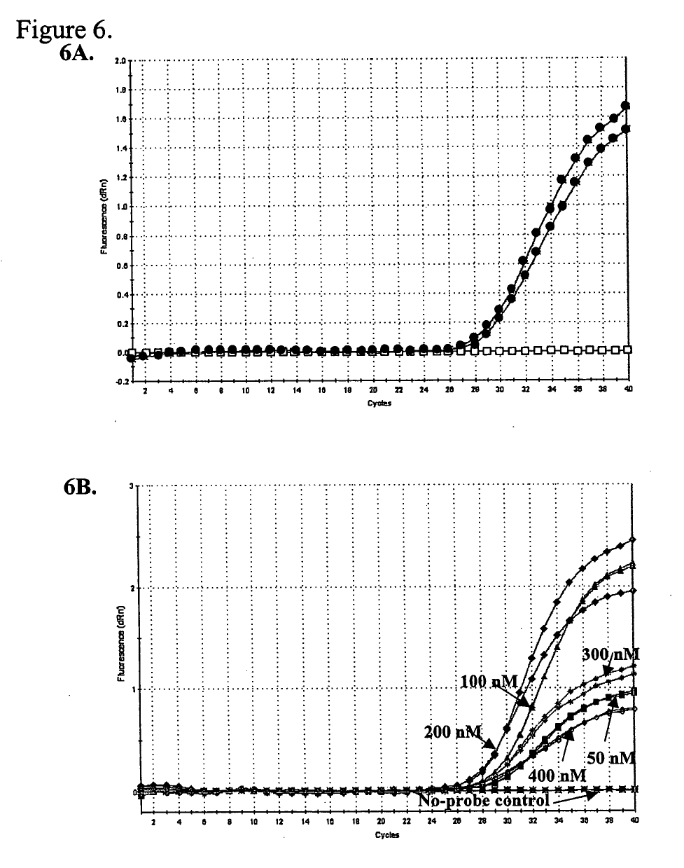

Fluorescence of the reaction was monitored over 40 cycles (FIG. 6A). Fluorescence of the amplification reaction (filled circles) was compared with a no probe controls (empty rectangles).

The concentration of the probe was titrated in a separate experiment. In this example, the concentration of the probe was varied. These experiments were performed essentially as described above. Amplification was carried out in a 50 μl total reaction volume, comprising, in addition to the probes:

1× FullVelocity buffer (containing dUTP instead of dTTP),

400 nM Forward and reverse GBS primers,

5 U Pfu V93R exo(-) polymerase,

200 ng FEN-1 endonuclease

30 nM ROX reference dye, and

5000 copies Streptococcus agalactiae genomic DNA as the template.

In this particular experiment, the triplex probe consisted of an equal molar ratio of the Quencher (3′BHQ1), Fluorophore (5°FAM), and BridgeS0 oligonucleotides. The Triplex probe concentrations tested were 50, 100, 200, 300, or 400 nM. Probe was omitted from the “No-probe control” reaction. Components were mixed together and thermo-cycled on the M×3000p real-time PCR instrument:

1 cycle at 95° C. for 2 minutes, followed by 40 cycles of:

95° C. for 1 second

60° C. for 18 seconds.

Fluorescence data was collected at the end of the 60° C. step at each cycle. The results, shown in FIG. 6B, indicate an ideal probe concentration of between about 100nM-200 nM using these probes.

### Example 3

### Quencher Oligonucleotide as Primer

The quencher oligonucleotides can be used as primers for PCR. As illustrated in FIG. 3, the quencher on the quencher oligonucleotide, when bridged next to the fluorophore oligonucleotide by hybridization to the bridging oligonucleotide, will act to quench the signal of the fluorophore on the fluorophore oligonucleotide. In addition, at least part of the quencher oligonucleotide sequence is complementary to the target DNA sequence. Therefore, under the right conditions of denaturation and annealing and in the presence of the target DNA, the quencher oligonucleotide can anneal to the target DNA sequence and serve as a template for DNA synthesis.

Conditions for performing the amplification reaction, as well as monitoring the reaction are as described in the previous examples. For example, an equal concentration of F, Q and bridging oligonucleotides can be used, preferably between 50 and 400 nM. Concentration of the primer can likewise be varied, from 50 to 400 nM. The concentrations of the primers can be titrated to provide optimal signal. Amplification is carried out in a 50 μl total reaction volume, comprising, in addition to the probes:

1× FullVelocity buffer (containing dUTP instead of dTTP),

400 nM Forward and reverse GBS primers,

5 U Pfu V93R exo(-) polymerase,

200 ng FEN-1 endonuclease

30 nM ROX reference dye, and

5000 copies Streptococcus agalactiae genomic DNA as the template.

### Example 4

### Bridging Oligonucleotide as Primer: Detection of CFTR

In this example, the bridging oligonucleotide, in addition to serving as a bridge to coordinate quenching of fluorescence from the fluorophore oligonucleotide, also serves a role as a primer for PCR. Using this approach, at least part of the bridging oligonucleotide is complementary to the target DNA sequence such that, under suitable conditions of denaturation and hybridization, the bridging oligonucleotide anneals to the region on the target DNA to which it has complementary sequence (See FIG. 4). In this variation the region complementary to the fluorophore and quencher oligonucleotides (‘P’) can be between 30 and 50 bases. When free in solution, the Fluorophore oligonucleotide (‘F’), Quencher oligonucleotide (‘Q’) and Bridge oligonucleotide hybridize together to form the triplex probe. As region ‘C.’ of the bridging oligonucleotide primes and incorporates into the amplicon, F and Q oligonucleotides stay bound. As the sense strand is primed by a second primer (‘D’) and extended beyond the region C, the quenched oligonucleotide Q′ is cleaved by nucleases (e.g., FEN nuclease), resulting in the liberation of the quencher or fluorophore moiety from either the fluorophore oligonucleotide or quencher oligonucleotide, resulting in enhanced fluorescence.

Detection of CFTR

In this example, CFTR was chosen as the gene target.

1. Probe

Bridging oligonucleotide: an ‘Alien’ sequence and the GBS probe recognition sequence were appended to the 5′ end of the CFTR_Rev primer ‘CFTR_Rev_Bridge’: 5′CGGCTTGGTCTGGCATGGAGGACAAGGGTTTGCGATGGTTCTGTTGTAGGTAGCAGTGGGCTGTAAACTCC3′ The region complementary to the ‘Q’ Oligonucleotide complement is highlighted in bold, and the region complementary to the ‘F’ Oligonucleotide is underlined.

Fluorescent oligonucleotide: the GBS probe with a Fam molecule on the 3′ end was used as the Fl Oligonucleotide. This probe is complementary to the 3′ half of the tag sequence on the reverse primer:

5′ TACCTACAACAGAACCATCGCAACCCT-FAM3′

Quencher oligonucleotide: Q Oligonucleotide: a BHQ1 molecule was appended to the 5′ end of an Alien probe complementary to the 5′ half of the tag sequence on the reverse primer:

5′ (BHQ1)-TGTCCTCCATGCCAGACCAAGCCG-PO₄ 3′

The primer for PCR;

CFTR_Fwd: 5′GCAGTGGGCTGTAAACTCC3′

A CFTR PCR product (1149 bp purified amplicon) was used as template for all testing. Detection of CFTR was performed using the following primer concentrations: 100 nM, 200 nM, or 300 nM each of the bridging oligonucleotide, fluorophore oligonucleotide and quencher oligonucleotide. The gene specific primer was used at 400 nM.

Detection reaction was carried out in a 50 ml QPCR reaction consisting of:

1× FullVelocity buffer

400 nM Forward and reverse GBS primers,

5 U Pfu V93R exo(-) polymerase,

200 ng FEN-1 endonuclease

30 nM ROX reference dye, and

100 fg Purified PCR template (114 bp amplicon ˜7.5×10⁵ copies).

Components were mixed together and thermo-cycled on the M×3000p real-time PCR instrument:

1 cycle at 95° C. for 2 minutes, followed by 40 cycles of:

95° C. for 10 Sec, and

60 C. 30 Sec

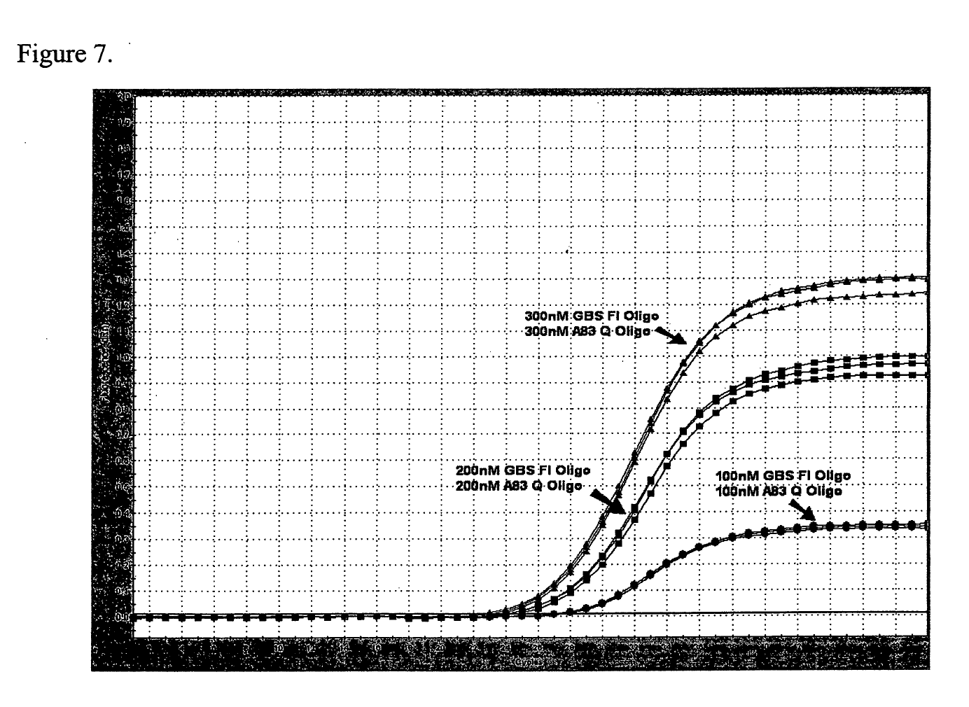

Results of the amplification of CFTR are shown in FIG. 7. Highest signal was generated using 300 nM of oligonucleotides.

### Example 5

### Bridging Oligonucleotide as Primer

This design (see FIG. 5) has the potential to show an increase in fluorescence unrelated to the amplification of the target nucleic acid. In this method, the tagged primer, free in solution, can out compete the quencher (Q) oligonucleotide for binding to the Bridge oligonucleotide, resulting in an increase in signal. This system can be used with the cycling conditions and reagent setup as previously described in Example 3, but the concentration of the Q oligonucleotide is used in excess of the Fluorescence and Bridging oligonucleotide concentrations and tagged primer to limit unwanted fluorescence when unincorporated tagged primer binds to the bridge oligonucleotide.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. An oligonucleotide probe complex, comprising:
a first oligonucleotide probe and a second oligonucleotide probe, wherein at least one of said first or second oligonucleotide probes binds to a target nucleic acid, wherein each of said first and second oligonucleotide probes comprise a member of an interactive pair of labels; and
a bridging oligonucleotide probe, wherein said bridging oligonucleotide probe binds to at least a portion of each of said first and second oligonucleotide probes, and wherein said members of the interactive pair of labels are in close proximity when bound to the bridging oligonucleotide probe.

2. The oligonucleotide probe complex of claim 1, wherein said interactive pair of labels comprises a fluorophore and a quencher.

3. The oligonucleotide probe complex of claim 2, wherein of one of said fluorophore or said quencher is attached to a 3′ nucleotide of said first oligonucleotide probe and the other of said fluorophore or said quencher is attached to a 5′ nucleotide of said second oligonucleotide probe.

4. The oligonucleotide probe complex of claim 1, wherein said first and/or second oligonucleotide probes have one or more nucleotides which do not hybridize to said bridging oligonucleotide.

5. The oligonucleotide probe complex of claim 1, wherein said interactive pair of labels are separated by between 0 and 5 nucleotides when bound to said bridging oligonucleotide.

6. The oligonucleotide probe complex of claim 2, wherein said fluorophore is selected from the group consisting of FAM, R110, TAMRA, R6G, CAL Fluor Red 610, CAL Fluor Gold 540, and CAL Fluor Orange 560.

7. The oligonucleotide probe complex of claim 2, wherein said quencher is selected from the group consisting of DABCYL, BHQ-1, BHQ-2, and BHQ-3.

8. The oligonucleotide probe complex of claim 1, wherein a detectable signal increases by at least 2 fold upon cleavage or hybridization of said first or second oligonucleotide probes to said target nucleic acid.

9. The oligonucleotide probe complex of claim 1, wherein a detectable signal increases by at least 3 fold upon cleavage or hybridization of said first or second oligonucleotide probes to said target nucleic acid.

10. A method of detecting a target nucleic acid in a sample, comprising the steps of:
contacting the sample with the oligonucleotide probe complex of claim 1; and determining the presence of the target nucleic acid in said sample, wherein a change in intensity of a signal is indicative of the presence of the target nucleic acid.

11. A method of detecting a target nucleic acid in a sample, comprising the steps of:
(1) providing a PCR mixture comprising the probe of claim 1, a nucleic acid polymerase, a nuclease and a pair of primers;
(2) contacting the PCR mixture with the sample to produce a PCR sample mixture; and
(3) incubating the PCR sample mixture of step 2, to allow amplification of the target nucleic acid and cleavage of said first and/or second oligonucleotide probes with said nuclease, wherein generation of a detectable signal is indicative of the presence of the target nucleic acid in said sample.

12. The method of claim 11, wherein said nucleic acid polymerase substantially lacks 5′ to 3′ exonuclease activity.

13. The method of claim 11, wherein the nucleic acid polymerase is a DNA polymerase.

14. The method of claim 11, wherein the said nuclease is a FEN nuclease.

15. The method of claim 11, wherein said nuclease and said nucleic acid polymerase are contained in a single enzyme.

16. A composition comprising the oligonucleotide probe complex of claims 1.

17. A kit for generating a signal indicative of the presence of a target nucleic acid sequence in a sample, comprising the oligonucleotide probe complex of claim 1 and packaging material therefor.

18. An oligonucleotide probe complex, comprising:
a first oligonucleotide probe and a second oligonucleotide probe, wherein at least one of said first or second oligonucleotide probes binds to a target nucleic acid, wherein each of said first and second oligonucleotide probes comprise a member of an interactive pair of labels and wherein said first and/or second probe binds to said target nucleic acid through a primer region; and
a bridging oligonucleotide probe, wherein said bridging oligonucleotide probe binds to at least a portion of each of said first and second oligonucleotide probes, and wherein said member of the interactive pair of labels are in close proximity when bound to the bridging oligonucleotide probe.

19. A composition comprising the oligonucleotide probe complex of claims 18.

20. A kit for generating a signal indicative of the presence of a target nucleic acid sequence in a sample, comprising the oligonucleotide probe complex of claim 18 and packaging material therefor.

21. A method of detecting a target nucleic acid in a sample, comprising the steps of:
(1) providing a PCR mixture comprising the probe of claim 18, a nucleic acid polymerase, and a primer;
(2) contacting the PCR mixture with the sample to produce a PCR sample mixture; and
(3) incubating the PCR sample mixture of step 2, to allow amplification of the target nucleic acid, wherein generation of a detectable signal is indicative of a presence of the target nucleic acid in said sample.

22. An oligonucleotide probe complex, comprising:
a first oligonucleotide probe and a second oligonucleotide probe, wherein each of said first and second oligonucleotide probes comprise a member of an interactive pair of labels; and
a bridging oligonucleotide probe, wherein said bridging oligonucleotide probe binds to, at least a portion of, each of said first and second oligonucleotide probes, wherein said bridging oligonucleotide probe binds to a target nucleic acid through a primer region, and wherein said member of the interactive pair of labels are in close proximity when bound to the bridging oligonucleotide probe.

23. A composition comprising the oligonucleotide probe complex of claims 22.

24. A kit for generating a signal indicative of the presence of a target nucleic acid sequence in a sample, comprising the oligonucleotide probe complex of claim 22 and packaging material therefor.

25. A method of detecting a target nucleic acid in a sample, comprising the steps of:
(1) providing a PCR mixture comprising the probe of claim 22, a nucleic acid polymerase, a nuclease and a primer;
(2) contacting the PCR mixture with the sample to produce a PCR sample mixture; and
(3) incubating the PCR sample mixture of step 2 to allow amplification of the target nucleic acid and cleavage of said first and/or second oligonucleotide probes with said nuclease, wherein generation of a detectable signal is indicative of the presence of the target nucleic acid in said sample.

26. An oligonucleotide probe complex, comprising:
a first oligonucleotide probe and a second oligonucleotide probe, wherein each of said first and second oligonucleotide probes comprise a member of an interactive pair of labels; and
a bridging oligonucleotide probe, wherein said bridging oligonucleotide probe, binds to at least a portion of each of said first and second oligonucleotide probes, wherein said bridging oligonucleotide probe binds to a target nucleic acid, and wherein said member of the interactive pair of labels are in close proximity when bound to the bridging oligonucleotide probe.

27. A composition comprising the oligonucleotide probe complex of claims 26.

28. A kit for generating a signal indicative of the presence of a target nucleic acid sequence in a sample, comprising the oligonucleotide probe complex of claim 26 and packaging material therefor.

29. A method of detecting a target nucleic acid in a sample, comprising the steps of:
(1) providing a PCR mixture comprising the probe of claim 26, a nucleic acid polymerase, a nuclease and a primer;
(2) contacting the PCR mixture with the sample to produce a PCR sample mixture; and
(3) incubating the PCR sample mixture of step 2, to allow amplification of the target nucleic acid and cleavage of said first and/or second oligonucleotide probes with said nuclease, wherein generation of a detectable signal is indicative of the presence of the target nucleic acid in said sample.


SEQUENCE LISTING (9 sequences)

SEQ ID NO: 1 - DNA, 42 bp, Artificial
  Bridging Oligonucleotide
ttgcgatggttctgttgtaggtcgcggcagggttctcgaggg

SEQ ID NO: 2 - DNA, 20 bp, Artificial
  Quenching  Oligonucleotide
ccctcgagaaccctgccgcg

SEQ ID NO: 3 - DNA, 26 bp, Artificial
  Fluorophore Oligonucleotide
acctacaacagaaccatcgcaaccct

SEQ ID NO: 4 - DNA, 71 bp, Artificial
  Bridge and Primer
cggcttggtctggcatggaggacaagggtttgcgatggttctgttgtaggtagcagtgggctgtaaactcc

SEQ ID NO: 5 - DNA, 27 bp, Artificial
  Fluorophore and probe sequence
tacctacaacagaaccatcgcaaccct

SEQ ID NO: 6 - DNA, 24 bp, Artificial
  Quencher Oligonucleotide
tgtcctccatgccagaccaagccg

SEQ ID NO: 7 - DNA, 19 bp, Artificial
  Forward Primer
gcagtgggctgtaaactcc

SEQ ID NO: 8 - DNA, 44 bp, Artificial
  Bridging Oligonucleotide
gggagctcttgggacggcgctttggatgttgtcttggtagcgtt

SEQ ID NO: 9 - DNA, 46 bp, Artificial
  Bridging Oligonucleotide
gggagctcttgggacggcgctttttggatgttgtcttggtagcgtt